(19) United States
(12) Patent Application Publication
(10) Pub. No.: US 2024/0266007 A1
(43) Pub. Date: Aug. 8, 2024

(54) INTEGRATED TOOL FOR AUTOMATICALLY GENERATING PROBABILITY BASED, NAT IONALLY AND STATE REPRESENTATIVE SAMPLES ACROSS THE GLOBE

(71) Applicant: Israel Terungwa Agaku, Flint, MI (US)

(72) Inventor: Israel Terungwa Agaku, Flint, MI (US)

(21) Appl. No.: 18/107,059

(22) Filed: Feb. 8, 2023

(51) Int. Cl. G16H 10/20

(52) U.S. Cl. CPC G16H 10/20

(57) ABSTRACT

This disclosure generally relates to a novel tool that has the potential to be deployed during public health emergencies and used by professionals with little to no expertise in survey methodology. Because sample sizes are estimated correctly and sampling executed speedily and validly, there can be more confident in the generated data for evidence-based decision-making.

### BACKGROUND

This invention relates generally to methods and systems for automatically generating probability and more particularly relates to an integrated tool for automatically generating probability-based, nationally and state representative samples across the globe.

### DESCRIPTION OF THE RELATED ART

Probability-based samples are the cornerstone of public health and policy. In the United States for examples, probability-based, nationally representative surveys such as the National Health and Nutrition Examination Survey (NHANES), are pivotal in tracking national objectives and benchmarks such as Healthy people 2030. Probability-based surveys are used to evaluate the impacts of public policies, to take the pulse of the nation on key matters (e.g., measuring extent of population support for certain policies), and to determine gaps in knowledge, practice and policy that could then inform tailored campaigns and other interventions. During public health emergencies, such as epidemics or pandemics, seroprevalence surveys are critical to measure the percentage of the population exposed to the contagion, to explore factors associated with exposure, and to determine which population groups have the greatest burden of exposure and disease.

In certain emergency contexts, surveys may need to be deployed rapidly in the span of just days or at most a few weeks. For example, the U.S. centers for Disease Control and Prevention (CDC) deploys members of the elite Epidemic Intelligence Service (EIS) and other career epidemiologists into hundreds of hot spots in the US and around the world where active outbreaks or other public health emergencies are happening. According to the Centers for Disease Control and Prevention, EIS officers have deployed and conducted rapid surveys at the scenes of public health emergencies including but not limited to Ebola, Zika, polio, Lassa fever, measles, COVID-19, and other emergencies.

Within such public health emergencies of grave consequence, it becomes imperative that data collection meets two key requirements to ensure an appropriate, timely, and evidence-based response: (1) Data collected must be fit for use and fit for purpose. (2) Data collection must be done speedily with the utmost urgency. These two requirements are discussed below, including current challenges to meeting both requirements, and how the innovation in this application helps to address the associated barriers.

### 1. Data Collected Must be Fit for Use and Fit for Purpose

For survey data collected during public health emergencies to be fit for use and purpose, the survey must be valid or generalizable. Validity means that the information obtained from the relatively small number of participants (“small” relative to the total size of the total population), must be able to be extrapolated to the broader population from which the sample was drawn. The basis for external validity or generalizability is random selection, which guarantees that everyone in the broader population has a known and non-zero probability of being selected. The use of volunteers or other non-probability-based sampling approaches may not be used to make any meaningful inferences from the sampled population to the target population because, lacking randomness, the characteristics of such participants is very unlikely to mirror that of the broader population.

2. Data Collection Must be Done Speedily with the Utmost Urgency.

Getting good quality after a significant delay may mean appropriate action is launched either late or too late, with significant implications in terms of lost lives, premature morbidity, direct medical expenditures and indirect costs from lost productively, amongst other societal tolls of such delayed evidence-based decision making. On the other hand, ‘quick but dirty’ data are equally dangerous as the quality of the decision making can only be as good as the quality of the data. Poor-quality data, being biased by virtue of its non-randomness, may have the unintended consequence of widening disparities if there is differential treatment of certain population subgroups based on policy decisions drawn from the flawed data.

These two data conditions of quality and speed must thus be met simultaneously. There therefore exists the need for a tool that can be used by professionals at the forefront of public health emergencies to synthesize evidence that instead of being ‘quick and dirty, is ‘rapid and useful’. To fill this gap, this application describes an innovation that allows for the automated generation of probability-based samples in every country of the world.

By looking at prior art multiple advancements have been seen in similar regards. For instance, a U.S. Pat. No. 7,653,509B2 on probability state models provides a method of analyzing multidimensional data using a computer as well as methods of displaying multidimensional data to a user for further analysis. The present invention provides for a method and system for state model Fitting. The system components include a detector and a computer operably connected to the detector. The computer accesses one or more logic instructions for receiving and analyzing raw data from the detector and generating a state model of the raw data based on probability weights.

A U.S. Pat. No. 6,954,722B2 relates to methods and systems for data analysis. The present invention provides methods of analyzing and/or displaying data. In one aspect, the invention provides methods for visualizing or displaying high dynamic range data obtained from flow cytometry analyses. Related systems and computer programs products are also provided.

A U.S. Pat. No. 10,609,173B2 relates to probability based caching and eviction. Some embodiments set forth probability based caching, whereby a probability value determines in part whether content identified by an incoming request should be cached or not. Some embodiments further set forth probability based eviction, whereby a probability value determines in part whether cached content should be evicted from the cache. Selection of the content for possible eviction can be based on recency and/or frequency of the content being requested. The probability values can be configured manually or automatically. Automatic configuration involves using a function to compute the probability values. In such scenarios, the probability values can be computed as a function of any of fairness, cost, content size, and content type as some examples.

A U.S. Pat. No. 11,475,343B2 relates to database utilizing spatial probability models for data compression. A method, article comprising machine-readable instructions and apparatus that processes data systems for encoding, decoding, pattern recognition/matching and data generation is disclosed. State subsets of a data system are identified for the efficient processing of data based, at least in part, on the data system's systemic characteristics.

A U.S. Pat. No. 9,612,669B2 relates to probability-based approach to recognition of user-entered data. A method for entering keys in a small key pad is provided. The method comprising the steps of: providing at least a part of keyboard having a plurality of keys; and predetermining a first probability of a user striking a key among the plurality of keys. The method further uses a dictionary of selected words associated with the key pad and/or a user.

A KR patent 1,022,64498B1 relates to computer program for predicting prevalence probability. According to an embodiment of the present disclosure, a computer program stored in a computer-readable storage medium is disclosed. When the computer program is executed on one or more processors, the computer program causes the one or more processors to perform the following operations for predicting prevalence probability. The operations comprise: an operation of receiving learning health data including learning state information and learning disease information; an operation of generating a prevalence probability prediction model by performing learning on an artificial neural network through the learning health data; an operation of calculating the prevalence probability of a user by using state information of the user, as an input of the prevalence probability prediction model; and an operation of generating health management information based on the prevalence probability and providing the generated health management information to a user terminal of the user.

By looking at prior art, multiple systems with their own features have been attempted. However, these solutions are limited and restricted to their conventional architecture, and installation system and have considerable shortcomings which adversely affect the convenience with which they can be used. The prior systems have certain limitations including the design of assemblies which make them not suitable for every type of use. Moreover, the ease-of-use ability of these systems is also questionable. The current invention proposes an advanced methodology of presenting an innovative system that allows for the automated generation of probability-based samples in every country of the world. It is a web-based application that is part of an ecosystem of tools known as Chisquares. This tool allows the entirety of the sample size calculation and probability proportional to size (PPS) sampling process to be accomplished within minutes for any country of the world, as well as for states within countries.

Thus it has been noted, to Applicant's knowledge, none of these prior art methods is entirely suitable to meet these needs and provide their own set of complexities. Thus, the present invention provides such a method, and the overall combination of these features is nowhere disclosed in the prior art cited above, which appears to represent the general art in this area, although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a complete description. A full appreciation of the various aspects of the invention can be gained by taking the entire specification, claims, drawings, and abstract as a whole.

The present subject matter relates generally to an improved system related to propose an advanced methodology of presenting an innovative system that allows for the automated generation of probability-based samples in every country of the world.

It is also the objective of the system to provide a web-based application that is part of an ecosystem of tools known as Chisquares. This tool allows the entirety of the sample size calculation and PPS sampling process to be accomplished within minutes for any country of the world, as well as for states within countries.

Another embodiment of the invention is that aspect, through an online system where the user has to select the country where the survey will be conducted, estimate their budget in US dollars, other countries and estimation of budgets in local currencies, and specify their mode of survey administration (e.g., in-person, phone or web-based), and indicate the main outcome from amongst hundreds provided via a drop-down menu.

Another objective of the system is to provide an online application that based on preset parameters, the tool automatically calculates the sample size needed for the survey based on the prevalence of the outcome.

It is accordingly an object of the invention to provide a dedicated web and client application that provides estimates of the number of counties and the number of people in each selected county that could feasibly be accessed based on the user's self-reported budget and mode of survey administration.

Another objective of the system is to provide a system that in particular, allows a system that probabilistically selects which counties have to be included in the survey, and the number of people that should be surveyed in each of the chosen counties. All the user must do thereafter is to use the output from the application to field the survey accordingly.

Although the invention is illustrated and described herein as embodied in a tool for automatically generating probability based on nationally and state samples, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

Other objects, features and advantages of the present invention will be apparent from the accompanying drawing and from the detailed description which follows.

This Summary is provided merely for purpose of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the algorithm for a web-based tool to streamline sample size and sampling operations to yield representative samples for individual countries around the globe incorporating the embodiments of the invention.

FIG. 2 shows the snapshot of the first 10 rows for the county database for Afghanistan and the United States as per preferred embodiments of the invention.

FIG. 3 shows the List of major areas to be covered in the prevalence database for the different countries as per preferred embodiments of the invention.

FIG. 4 shows the login screen per preferred embodiments of the invention.

FIG. 5 shows the registration form as per preferred embodiments of the invention.

FIG. 6 shows another form interface as per preferred embodiments of the invention.

FIG. 7 shows the sign-in form for returning users as per preferred embodiments of the invention.

FIG. 8 shows the forgotten password form as per preferred embodiments of the invention.

FIG. 9 shows the email received in case of a forgotten password as per preferred embodiments of the invention.

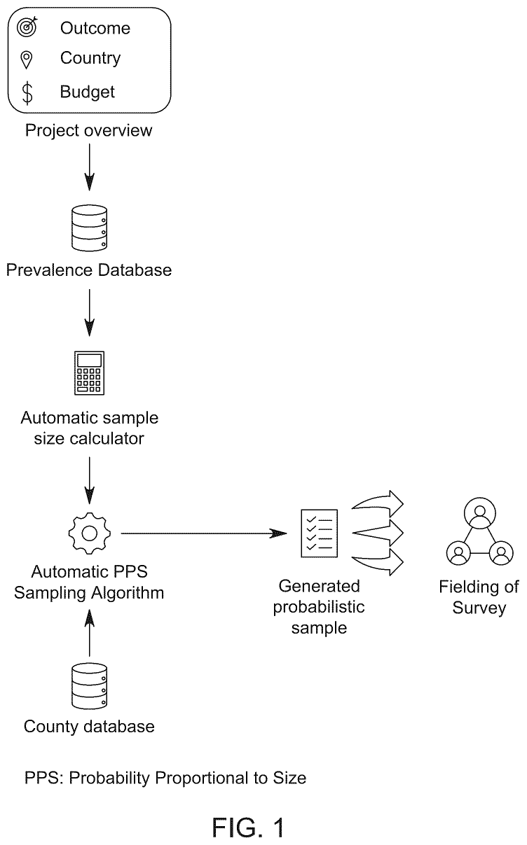

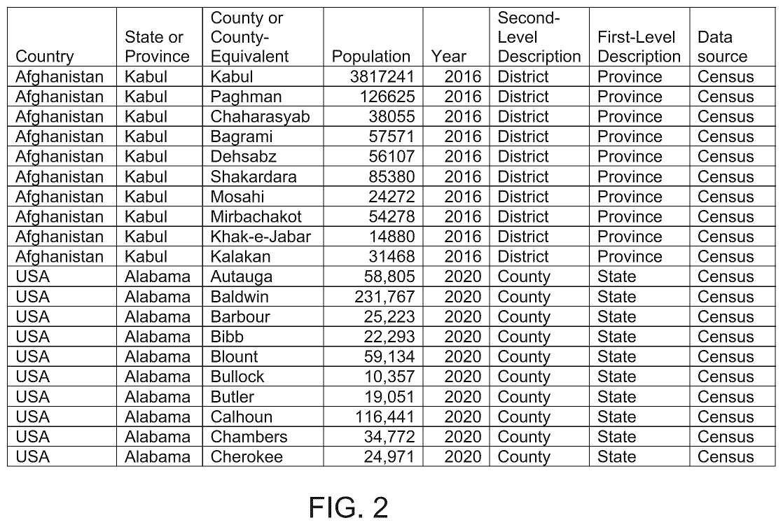

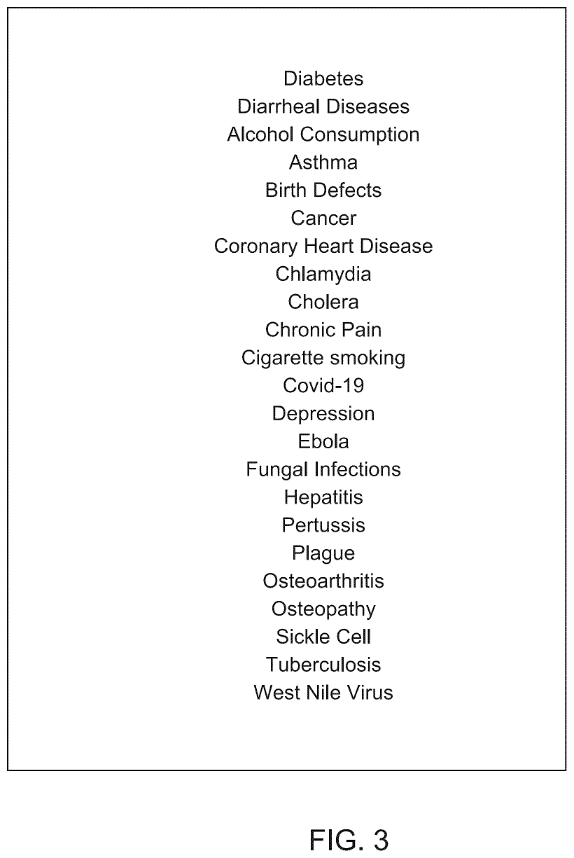

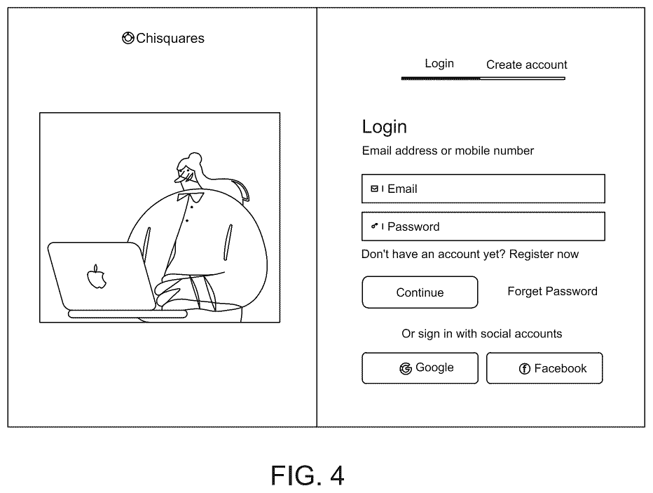

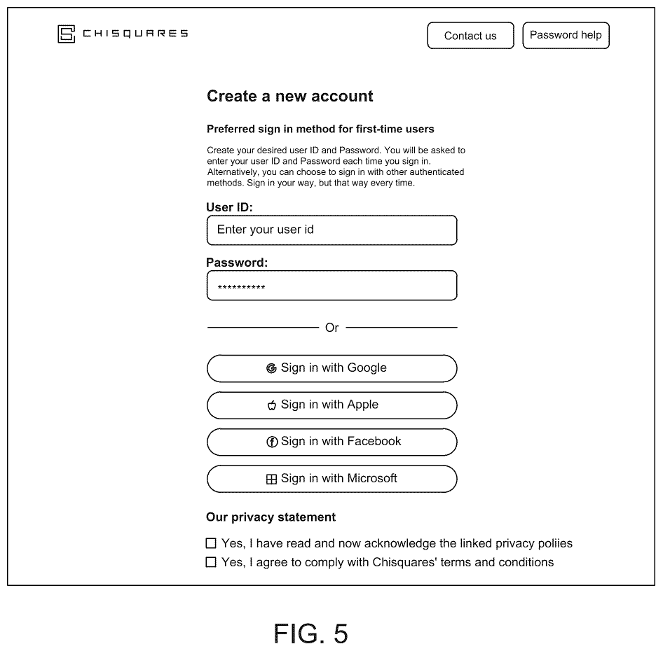

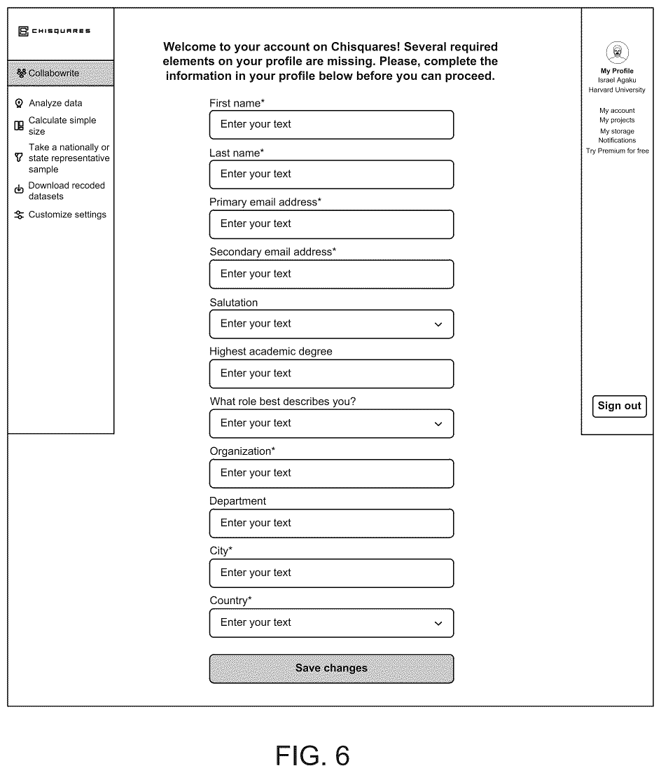

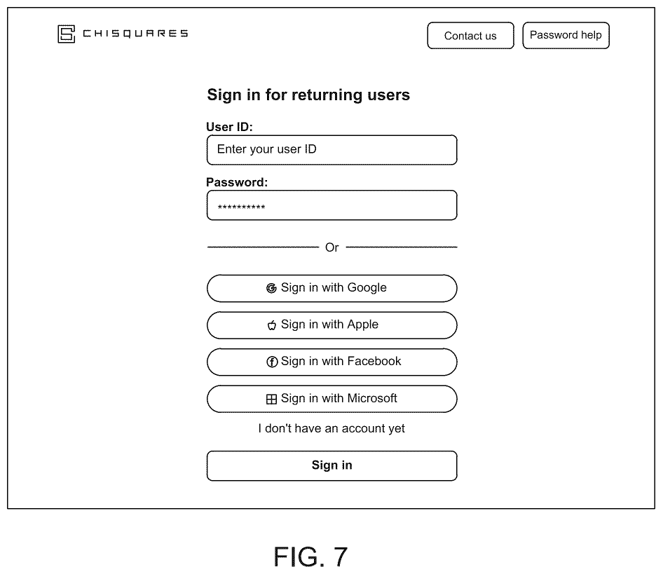

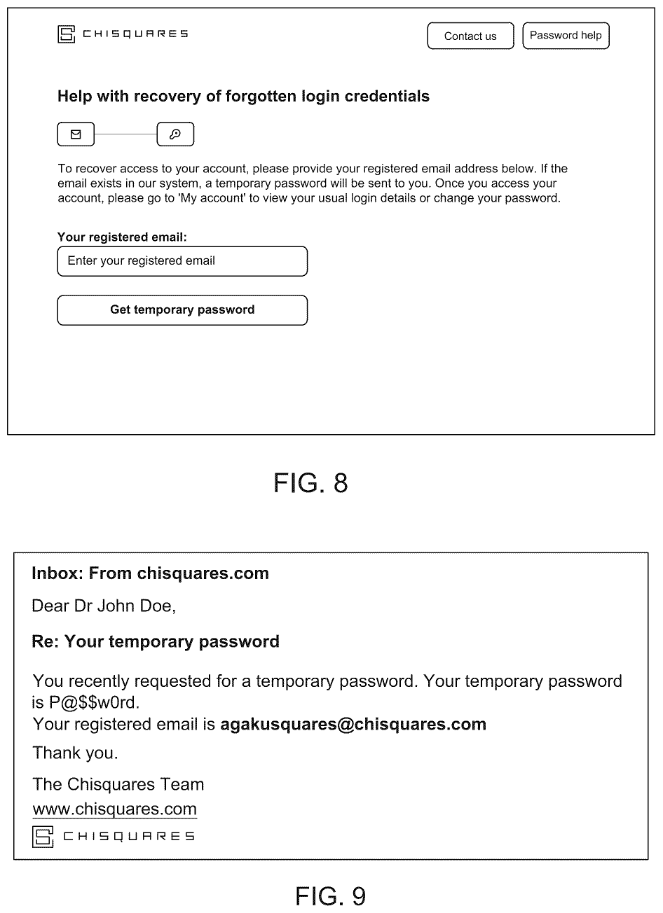

The apparatus and method components have been represented where appropriate by conventional symbols in the drawings, showing only those specific details

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. Accordingly, the present invention may take the form of an entirely hardware embodiment, an entirely software embodiment or an embodiment combining software and hardware aspects. The following detailed description is, therefore, not to be taken in a limiting sense.

A web-based application as per its preferred embodiments is proposed that is part of an ecosystem of tools known as Chisquares. This tool allows the entirety of the sample size calculation and PPS sampling process to be accomplished within minutes for any country of the world, as well as for states within countries. All the user has to do is select the country where the survey will be conducted, estimate their budget in US dollars or resources for the study, specify their mode of survey administration (e.g., in-person, phone or web-based), and indicate the main outcome from amongst hundreds provided via a drop-down menu. Based on these parameters, the tool automatically calculates the sample size needed for the survey based on the prevalence of the outcome and estimates the number of counties that could feasibly be accessed based on the user's self-reported budget and mode of survey administration. Furthermore, the tool probabilistically selects which counties have to be included in the survey, and the number of people that should be surveyed in each of the chosen counties. All the user must do thereafter is to use the output from the application to field the survey accordingly.

The web application as per its further embodiments, has the following critical components. An information gathering component that collects information from the user on the particulars of their survey, including the key outcome, the country where it is being conducted, their budget for the total survey (will determine how many counties will be sampled), and the mode of survey administration. Publicly available records from past and ongoing surveys in the U.S. (obtained from Parts A and B of regulatory submissions sent to the Office on Management and Budget for approval as required by the paperwork Reduction Act), will be used to estimate the incremental cost of adding one more county to a national survey (using a linear regression model). This estimate will serve as a benchmark to estimate how many counties a given budget could support, considering the mode of data collection (e.g., face to face, or telephone), which have different associated implementation costs in the real world.

As per its further embodiments, a large database containing prevalence estimates for hundreds of different outcomes for all countries of the world separately. This database will be curated from authoritative sources of information such as ‘Our World in Data’; official reports from the World Health Organization, the United Nations, and federal Government reports; scientific articles; white papers; and several other reviewed sources. This database will provide the prevalence estimates to be input for sample size calculation for each country of interest.

As per its further embodiments, a large database comprising an exhaustive list of all the counties or county-equivalents for all countries of the world, along with their most recent census estimates or projections is provided. This is a critical part of the platform as the counties or county-equivalents are the clusters of interest and their populations provide a measure of size for PPS sampling. The manner in which these county data are compiled and organized will allow the user to fetch a representative sample at either the national level or at the level of individual states for any given country.

As per its further embodiments, the output from the platform will be in the form of an Excel spreadsheet which will contain the following elements:
Column A: the selected counties or county-equivalents
Column B: the population of each of the counties selected
Column C: the probability of selection at the first stage of sampling (i.e., the primary sampling unit, or the county level).
Column D: The number of people to be selected in each county that was sampled.
Column E: The probability of selection at the second stage of sampling (i.e., individual-level sampling).
Column F: The joint probability of selection accounting for both the first and second stages of sampling.

The sampling approach utilized in the web application (i.e., a two-stage cluster sample) is designed to yield a self-weighting sample to eliminate the need for front-line workers to worry about creating complicated weighting algorithms to correct for differential probabilities of selection. This self-weighting attribute is achieved by favoring the bigger clusters in the first stage of sampling but favoring the smaller clusters in the second stage of sampling. Specifically, because the probability of selection is proportional to size in the first stage of selection, bigger clusters are more likely to be selected than smaller ones. This bias is however evened out in the second stage of selection by selecting a fixed number of participants from each county selected, regardless of the size of the county, thus ensuring individuals in smaller counties have a higher probability of selection. As will be seen from Column F of the output, this counterbalancing two-stage approach creates a self-weighting sample where the probability of selection for everyone in the sample is equal.

It is appreciated that additional advantages, modifications and equivalent embodiments will be apparent to those skilled in the art. Therefore, the invention, in its broader aspects, is not limited to the specific details shown and described herein. Accordingly, various modifications may be made without departing from the spirit or scope of this invention as defined by the appended claims and their equivalents.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The invention claimed is:

1. An algorithm for a web-based tool to streamline sample size and sampling operations to yield representative samples for individual countries around the globe wherein:
The system as per claim 1, calculates the sample size needed for the survey based on the prevalence of the outcome;
The system as per claim 1, estimates the number of counties that could feasibly be accessed based on the user's self-reported budget and mode of survey administration,
The system as per claim 1, probabilistically selects which counties have to be included in the survey, and the number of people that should be surveyed in each of the chosen counties. All the user must do thereafter is to use the output from the application to field the survey accordingly.

2. A survey tool that includes components:
As per claim 2, the information gathering component that collects information from the user on the particulars of their survey, including the key outcome, the country where it is being conducted, their budget for the total survey (will determine how many counties will be sampled), and the mode of survey administration;
As per claim 2, the Publicly available records from past and ongoing surveys in the U.S. (obtained from Parts A and B of regulatory submissions sent to the Office on Management and Budget for approval as required by the paperwork Reduction Act), will be used to estimate the incremental cost of adding one more county to a national survey (using a linear regression model);
As per claim 2, an estimate will serve as a benchmark to estimate how many counties a given budget could support, considering the mode of data collection (e.g., face to face, or telephone), which have different associated implementation costs in the real world;
As per claim 2, a large database containing prevalence estimates for hundreds of different outcomes for all countries of the world separately;
As per claim 2, the database will be curated from authoritative sources of information such as ‘Our World in Data’; official reports from the World Health Organization, the United Nations, and federal Government reports; scientific articles; white papers; and several other reviewed sources;
As per claim 2, the database will provide the prevalence estimates to be input for sample size calculation for each country of interest;
As per claim 2, the large database comprises an exhaustive list of all the counties or county-equivalents for all countries of the world, along with their most recent census estimates or projections.

3. An integrated tool for automatically generating probability-based, nationally and state-representative samples across the globe where the output includes:
As per claim 3, the selected counties or county-equivalents;
As per claim 3, the population of each of the counties selected;
As per claim 3, the probability of selection at the first stage of sampling (i.e., the primary sampling unit, or the county level);
As per claim 3, the number of people to be selected in each county that was sampled;
As per claim 3, the probability of selection at the second stage of sampling (i.e., individual-level sampling);
As per claim 3, the joint probability of selection accounts for both the first and second stages of sampling.
